(19) United States
(12) Patent Application Publication
(10) Pub. No.: US 2011/0251630 A1
(43) Pub. Date: Oct. 13, 2011

(54) CORNEAL MARKING APPARATUS

(76) Inventors: Gary A. Richardson, Rochester, NY (US); Ronald Spoor, Peen Yan, NY (US); Robert Stupplebeen, Webster, NY (US); Kevin Newman, Rochester, NY (US); Rolf Meyer, Port (CH)

(21) Appl. No.: 12/756,393

(22) Filed: Apr. 8, 2010

(51) Int. Cl. A61F 9/007

(52) U.S. Cl. 606/166

(57) ABSTRACT

A handheld instrument for marking a cornea, comprising: a handle; a pendulum coupled to the handle; an eye contact element coupled to the pendulum, the eye contact element comprising an eye contact surface; and a corneal marker comprising a marking element, the corneal marker being rotatably coupled to the pendulum. The marking element is disposed within the eye contact element at a position such that the marking element is capable of contacting a cornea when the eye contact surface is in contact with the eye. The eye contact element may be retractably coupled to the pendulum, the eye contact element being retractable from a position at which the marking element is disposed within the eye contact element and cannot contact the cornea, to a retracted position at which the marking element is disposed at said position where the marking element is capable of contacting a cornea when the eye contact surface is in contact with the eye

### FIELD OF INVENTION

The present invention relates to corneal marking apparatus.

### BACKGROUND OF THE INVENTION

Astigmatism is an optical defect which blurs vision due a non-rotationally symmetric curvature of the cornea of the eye. In individuals with astigmatism, the cornea is curved in a manner similar to the surface of a football, with the ball being curved such that the surface has a greater curvature in a steep meridian that extends through the center of the eye than the curvature in a shallow meridian that extends through the center of the eye. This causes a blurring of light rays from an object because light is focused differently depending on the meridian in which the rays are located.

Astigmatism is measured in terms of a cylinder power and axis. The cylinder power is measured as the difference in diopters between the optical power in the steep meridian and the optical power in the shallow meridian, and the axis is measured as the angular deviation of the steep meridian from horizontal.

The goal of treating astigmatism is to address the uneven curvature that causes blurred vision. Astigmatism may be corrected with eyeglasses, contact lenses and/or surgery. Surgical correction of astigmatism can include laser surgery (such as LASIK) as well as LRI (Limbal Relaxing Incisions) and implantation of toric intraocular lenses (IOLs).

Intraocular lenses are artificial lenses typically used to replace natural crystalline lenses of patients' when their natural lenses are diseased or otherwise impaired. During IOL implantation, astigmatic error can be corrected using a toric IOL, possibly in combination with performing Limbal Relaxing Incisions in the peripheral cornea to flatten the steeper meridian.

During surgery, regardless of whether astigmatism is corrected with a laser, LRI or with a toric IOL, it is desirable to align the surgical intervention according to the orientation of the steep and shallow meridians. Accordingly, prior to the surgery it is typically desirable to mark the orientation of a meridian onto a patient's cornea as accurately as possible.

There is a need for an accurate and convenient system and method for marking an astigmatic axis on the cornea.

### SUMMARY

Aspects of the present invention are directed to a handheld instrument for marking a cornea. The instrument comprises a handle; a pendulum coupled to the handle; an eye contact element coupled to the pendulum, the eye contact element comprising an eye contact surface; and a corneal marker comprising a marking element, the corneal marker being rotatably coupled to the pendulum. The marking element is disposed within the eye contact element at a position such that the marking element is capable of contacting a cornea when the eye contact surface is in contact with the eye.

The eye contact element may be retractably coupled to the pendulum, the eye contact element being retractable from a position at which the marking element is disposed within the eye contact element and cannot contact the cornea, to a retracted position at which the marking element is disposed at said position where the marking element is capable of contacting a cornea when the eye contact surface is in contact with the eye

In some embodiments, the instrument having an instrument axis extends through the eye contact element and the cornea marker, such that the instrument provides a view of the eye along the instrument axis when the corneal contact surface is in contact with the eye.

In some embodiments, the instrument is configured to permit the pendulum to rotate only in a single plane. In some embodiments, the pendulum comprises a ballast and a shaft, both of which are rotatable relative to the handle, the corneal marker being rigidly coupled to the ballast by the shaft.

In some embodiments, the handle comprises a frame portion to which the pendulum is coupled. The frame portion may form a closed figure.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic, projection view of an example of a handheld corneal marking instrument according to aspects of the present invention;

FIG. 2 is a cross-sectional view of the handheld corneal marking instrument shown in FIG. 1;

FIG. 3 is a schematic, projection view of another example of a handheld corneal marking instrument according to aspects of the present invention; and

FIG. 4 is a cross-sectional view of the handheld corneal marking instrument shown in FIG. 3.

### DETAILED DESCRIPTION

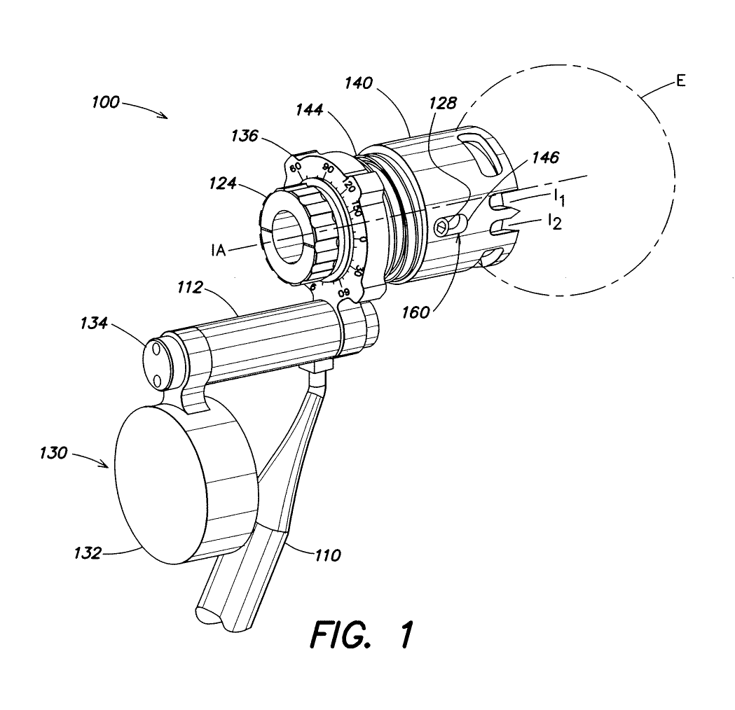

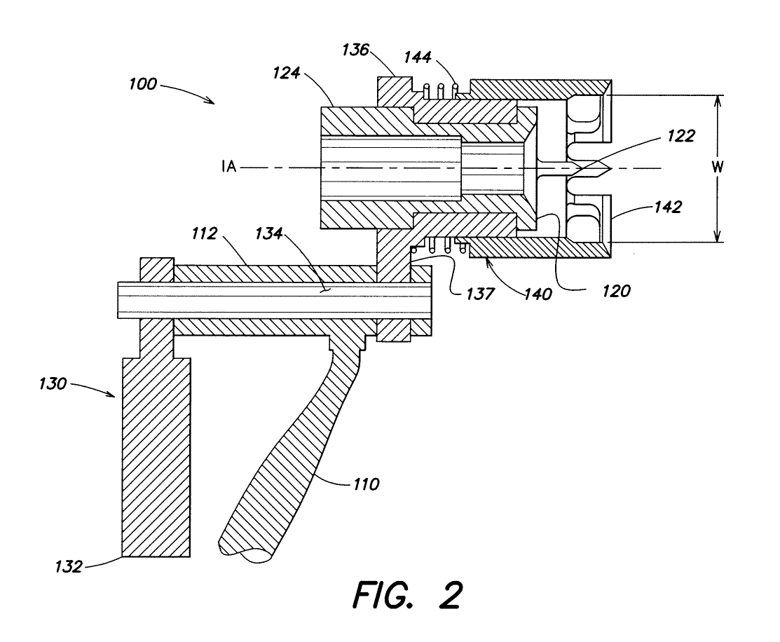

FIGS. 1 and 2 are projection and cross-sectional views, respectively, of an example of a handheld corneal marking instrument according to aspects of the present invention. Instrument 100 comprises a handle 110 (shown in-part in FIGS. 1 and 2), a corneal marker 120, a pendulum 130 for aligning corneal marker 120 using gravity, and an eye contact element 140 for stabilizing the instrument relative to an eye E.

Handle 110 is configured to permit a user to maneuver instrument 100 so as to align it with an eye E and bring the instrument into contact with the eye E. The handle may have any suitable shape to maneuver the instrument. For example, the handle may be a simple cylindrical pole with a textured surface for gripping. The handle may also include one or more connector portions 112 for connecting to one or more of the other portions of instrument. The handle may be angled to reduce interference of the instrument with a patient's face (e.g. brow, nose and/or cheek.

Pendulum 130 is coupled to handle 110 to allow rotation of the pendulum relative to the handle such that the pendulum can align with gravity. In the illustrated embodiment, the rotation occurs in a single plane, although in other embodiments other degrees of freedom may be possible to align with gravity. In the illustrated embodiment, pendulum 130 comprises three components a ballast 132, a shaft 134 and a flange 136. The shaft extends through tubular connector portion 112 of the handle and is rotatable within the tubular portion. Flange 136 is coupled to the ballast 132 by shaft 134 such that both the ballast and the flange are rotatable relative to the handle. It will be appreciated that such a configuration allows the ballast to extend downward to align with gravity and allows the flange (which is of lesser weight than ballast 132) to extend upward to align with gravity.

Corneal marker 120 is rotatably coupled to the pendulum. In particular, in the illustrated embodiment, corneal marker 120 is connected to flange 136. The corneal marker comprises a dial portion 124 that is accessible to a user of the instrument. By grasping and turning the dial portion, the corneal marker can be rotated relative to the pendulum to achieve an angular offset relative to gravitationally-determined alignment of the pendulum to achieve an offset from horizontal. In some embodiments, a portion of the pendulum (e.g., flange 126) is provided with indicia indicating a relative angular alignment between corneal marker and the pendulum.

At its distal end, corneal marker 120 comprises a marking element 122 that is configured to contact eye E so as to make a visible mark on the corneal surface. For example, the marking element can be configured and arranged to indent the cornea by pressing against the cornea or marking element 122 can be provided with an ink suitable for temporarily marking the cornea. In some embodiments, the marking element can be sharp so as to cut the epithelium, heated to denature the cornea and/or adapted to vibrate to facilitate marking.

Eye contact element 140 comprises an eye contact surface 142. The contact surface is shaped to provide a relatively comfortable and stable interface with the eye. The eye contact element is retractably coupled to pendulum 130. In the illustrated embodiment, the eye contact element is slidably coupled with flange 126, and a spring 144 is provided between a flange surface 137 and eye contact element 140 to provide a resistance to sliding when the eye contact element contacts eye E. The contact surface is sized to touch portions of the eye that are disposed radially outward of the limbus for most of the population. For example, a width W of the marking element (e.g., the inner diameter of a circular marking element) may be in the range 10-12.5 mm. Also, the eye contact element may be formed with multiple interruptions I₁ and I₂ in the eye contact surface to increase traction of the eye contact surface on the eye. Additionally, the eye contact surface may have a bevel on it to approximate the curvature of the eye to increase comfort and contact area on the eye.

Eye contact element 140 is retractable from a first position (shown in FIG. 2) at which marking element 122 is disposed within eye contact element 140 and cannot contact the cornea, and a fully retracted position (not shown; i.e., where spring 144 is compressed) at which the marking element 122 is disposed within eye contact element 140 and can contact the cornea of eye E. It will be appreciated that, due to the spheroid shape of the cornea, when the eye contact surface 142 contacts the eye, a portion of the cornea will extend into eye contact element 140 thereby permitting contact of marking element 122 with the corneal surface of eye E when the eye contact element 140 is in a position to mark an eye (i.e., in a fully or partially retracted position). Accordingly, the eye contact element provides stability of the instrument as determined by eye contact surface 142 and provides safety by keeping the marking element from protruding from the eye contact element (even when the eye contact element is in its fully retracted position) and risking damage to the eye caused by the relatively sharp marking element. Additionally, in some embodiments, it is advantageous to provide an alignment apparatus 160 such that the corneal marker can translate relative to the eye contact element without rotation. Accordingly, in the illustrated embodiment, eye contact element 140 is provided with one or more slots 146 and the corneal marker is provided with a corresponding one or more pins 128 that extend into the slots 146 and travel along the length of the slots. The one or more pins are sized to prevent motion along the widths of the slots (i.e., to prevent rotation of the corneal marker).

Although in the illustrated embodiment the eye contact element is retractable, the instrument need not be so configured. The eye contact element may be non-retractable. In such embodiments, the marking element is disposed within the eye contact element at a position such that the marking element (which is at a fixed axial location relative to the eye contact element) is capable of contacting a cornea when the eye contact surface is in contact with the eye. In such embodiments, the end of the marking element may be flush with eye contact surface 142 or recessed within eye contact element 140. It will be appreciated that the distance which the marking element is recessed within the eye contact element to be able to contact the cornea is determined by the width W and the fact that a typical eye has a diameter of about 24 mm.

Instrument 100 has an instrument axis IA that extends through eye contact element 140 and the cornea marker 120. The eye contact element and the cornea marker are hollow such that, when corneal contact surface 142 is in contact with eye E, a view of the eye is provided to a user along the instrument axis. Such a configuration facilitates alignment of instrument 100 with the eye as the instrument is brought into contact with the eye. In some embodiments, portions of the instrument that contact the eye are disposable. For example, said portions can be made of a plastic.

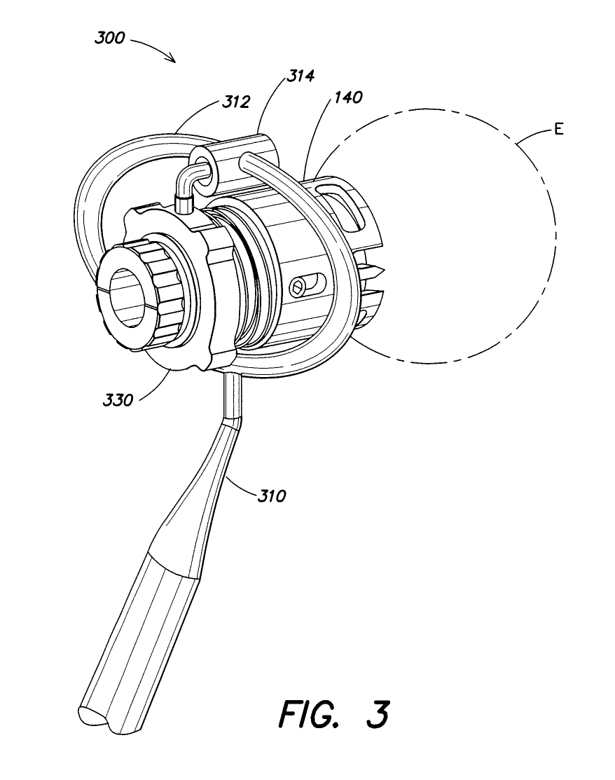

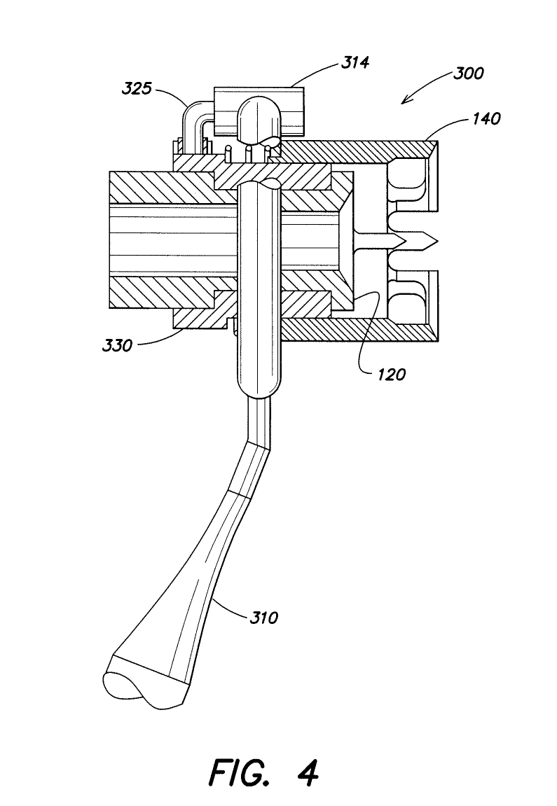

FIGS. 3 and 4 are a projection view and a cross-sectional view, respectively, of another example of a handheld corneal marking instrument 300 according to aspects of the present invention. Similar to instrument 100 described above, instrument 300 comprises a handle 310, a corneal marker 120, a pendulum 330 for aligning corneal marker 120 using gravity, and an eye contact element 140 for stabilizing the instrument relative to eye E. However, in instrument 300, handle 310 comprises a frame portion 312 to which pendulum 330 is coupled, and a coupling structure 325 used to couple pendulum 330 to frame portion 312.

The pendulum is coupled to the frame in a manner that allows rotation of the pendulum relative to the handle such that the pendulum can align with gravity. The frame is selected to be large enough so that it does not impair the rotational movement, provided said movement is less than a selected maximum. It will be appreciated that a maximum can be selected based on the expected amount handle 310 will deviate from a vertical position during use. In the illustrated embodiment, the frame forms a closed oval shape. However, any suitable shape may be used (e.g., circle, oval or polygonal) and the shape may form a closed figure or an open figure.

In instrument 300, the pendulum to which the corneal marker and eye marking apparatus are coupled is coupled to the frame using an elbow-shaped coupling structure 325 that extends into a flange 314 on frame portion 312. Typically, coupling structure 325 and flange 314 will be configured to permit only rotational movement of the pendulum (and the eye contact element 140 and corneal marker 120) within a plane (i.e., the elbow is only able to spin about its center axis). However, in some embodiments some additional movement that allows alignment with gravity may be permitted.

In a typical use, the handheld instrument is grasped by its handle and the pendulum is allowed to self level (to determine horizontal). The corneal marker, which is coupled to the pendulum, is rotated to orient the eye marking element with a previously measured deviation from horizontal. Next, the eye contact element is brought into contact with a portion of the eye that is disposed radially outward of the limbus of the eye. At contact, the eye contact element is in a first position at which the marking element is disposed within the eye contact element and cannot contact the cornea. Subsequently, the instrument is moved forward (i.e., toward the eye so as to press against the eye), such that the eye contact element is retracted relative to the marking element. The eye contact element thus achieves a fully or partially retracted position at which the marking element is disposed within the eye contact element at a position at which it can contact the cornea, thereby marking the cornea.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. A handheld instrument for marking a cornea, comprising:
a handle;
a pendulum coupled to the handle;
an eye contact element coupled to the pendulum, the eye contact element comprising an eye contact surface; and
a corneal marker comprising a marking element, the corneal marker being rotatably coupled to the pendulum,
the marking element is disposed within the eye contact element at a position such that the marking element is capable of contacting a cornea when the eye contact surface is in contact with the eye.

2. The instrument of claim 1, wherein the eye contact element is retractably coupled to the pendulum and wherein the eye contact element is retractable from a position at which the marking element is disposed within the eye contact element and cannot contact the cornea, to a retracted position at which the marking element is disposed at said position where the marking element is capable of contacting a cornea when the eye contact surface is in contact with the eye.

3. The instrument of claim 1, wherein the instrument having an instrument axis extending through the eye contact element and the cornea marker, the instrument providing a view of the eye along the instrument axis when the corneal contact surface is in contact with the eye.

4. The instrument of claim 1, wherein the instrument is configured to permit the pendulum to rotate only in a single plane.

5. The instrument of claim 1, wherein the pendulum comprises a ballast and a shaft, both of which are rotatable relative to the handle, the corneal marker being rigidly coupled to the ballast by the shaft.

6. The instrument of claim 1, wherein the handle comprises a frame portion to which the pendulum is coupled.

7. The instrument of claim 6, wherein the frame portion forms a closed figure.
